(19) United States
(12) Patent Application Publication
(10) Pub. No.: US 2016/0367311 A1
(43) Pub. Date: Dec. 22, 2016

(54) Instrumentation with Embedded Imaging Systems

(71) Applicant: Lawrence J. Gerrans, San Anselmo, CA (US)

(72) Inventor: Lawrence J. Gerrans, San Anselmo, CA (US)

(21) Appl. No.: 14/740,953

(22) Filed: Jun. 16, 2015

(51) Int. Cl. A61B 18/14; A61B 5/145; A61B 5/00; A61B 10/02

(52) U.S. Cl. CPC A61B 18/1445; A61B 18/1482; A61B 10/02; A61B 5/14503; A61B 5/0084; A61B 2217/007; A61B 2018/00982

(57) ABSTRACT

A medical instrument includes a shaft having a proximal end, a distal end and a longitudinal axis, and a working member at the distal end of the shaft and having a first arm and a second arm, wherein each of the first and second arms has a proximal end and a distal end, and wherein at least of the first and second arms pivots relative to the longitudinal axis of the shaft. The medical instrument further includes a first diagnostic device positioned in the first arm adjacent the distal end of the first arm, a second diagnostic device positioned in the second arm adjacent the distal end of the second arm, and a third diagnostic device positioned adjacent the proximal ends of the first and second arm.

### FIELD OF THE INVENTION

The present invention relates to medical instruments having imaging capabilities. More specifically, the present invention relates to a medical instrument having a plurality of integrated imaging and/or diagnostic devices.

### BACKGROUND OF THE INVENTION

There are many medical devices currently available for performing a variety of medical procedures. Typically, the medical devices are used together with a separate endoscope or another type of an imaging device to visualize the medical procedure in vivo. However, such devices suffer from a number of drawbacks. First, the need for insertion of a separate imaging device makes the procedure more complex, requiring additional steps, bulkier working channels, and larger incisions.

Additionally, the imaging devices often get foggy or dirty because of contact with various bodily tissues and fluids, which impairs their imaging capabilities, and need to be withdrawn from the patient's body to be cleaned and then reinserted. This makes the procedure more complicated and traumatic for the patient.

Furthermore, often it is desirable to perform diagnostic tests simultaneously with a medical procedure. In order to perform such tests, a separate diagnostic device needs to be inserted into the patient's body, in addition to the working medical device and the imaging device. Again, this leads to longer and more complicated procedures, requiring a physician to insert and operate multiple devices, and also makes the procedure more difficult for the patient.

What is desired, therefore, is a medical instrument, such as a biopsy device, that can also perform diagnostic functions to eliminate the need for a separate procedure. What is also desired is a medical device with an imaging system that facilitates proper insertion of the medical device into a bodily cavity and assists in performing a medical procedure via the medical device. What is further desired in a medical device with imaging and/or diagnostic capabilities that includes a cleaning system that allows a user to efficiently clean the imaging/diagnostic device lenses without the need to remove the device from a patient's body. It is also desired to provide a medical device with imaging and/or diagnostic capabilities that is more cost effective, reusable with various existing devices, and simpler in design.

### SUMMARY OF THE INVENTION

Therefore, it is an objective of this invention to provide a medical instrument that performs a medical procedure, such as biopsy, and also performs diagnostic functions to eliminate the need for a separate procedure.

It is also an objective of this invention to provide a medical device with an imaging system that facilitates proper insertion of the medical device into a bodily cavity and assists in performing a medical procedure via the medical device.

It is further an objective of this invention to provide a medical device with imaging and/or diagnostic capabilities that includes a cleaning system that allows a user to efficiently clean the imaging/diagnostic device lenses without the need to remove the device from a patient's body.

It is yet a further objective of the present invention to provide a medical device with imaging and/or diagnostic capabilities that is more cost effective, reusable with various existing devices, and simpler in design.

In order to overcome the deficiencies of the prior art and to achieve at least some of the objectives and advantages listed, the invention comprises a medical instrument, including a shaft having a proximal end, a distal end and a longitudinal axis, a working member at the distal end of the shaft and having a first arm and a second arm, wherein each of the first and second arms has a proximal end and a distal end, and wherein at least one of the first and second arms pivots relative to the longitudinal axis of the shaft, a first diagnostic device positioned in the first arm adjacent the distal end of the first arm, a second diagnostic device positioned in the second arm adjacent the distal end of the second arm, and a third diagnostic device positioned adjacent the proximal ends of the first and second arm.

In some embodiments, at least one of the first arm and the second arm has a cutting surface. In additional embodiments, at least one of the first arm and the second arm comprises a grasping surface.

In certain embodiments, at least one of the first arm and the second arm has a storage compartment for retaining a tissue sample.

In some embodiments, the working member includes an electrically conductive member coupled to a source of electrical current for delivering electric current to tissue.

In some cases, the working device is removably attached to the shaft.

In certain embodiments, the medical instrument further includes an actuator positioned at the proximal end of the shaft that actuates the working device.

In some embodiments, at least one of the first, second and third diagnostic devices has a sensor that detects and measures at least one characteristic of bodily tissue.

In certain advantageous embodiments, at least one of the first, second and third diagnostic devices is a camera. In some of these embodiments, the camera has at least one lens and at least one imaging sensor. In certain of these embodiments, the imaging sensor is a CMOS sensor.

In some embodiments, at least one of the first, second and third diagnostic devices further includes at least one illumination device positioned adjacent the camera. In certain of these embodiments, the at least one illumination device has a light source emitting light with a visible spectrum. In additional embodiments, the at least one illumination device has a light source emitting light with a non-visible spectrum.

In certain embodiments, the medical instrument further includes a processor connected to the first, second and third diagnostic devices that receives and processes data captured by the diagnostic devices. In some of these embodiments, the processor is connected to the first, second and third diagnostic devices via a wired connection. In additional embodiments, the processor is connected to the first, second and third diagnostic devices via a wireless connection.

In some cases, the working device includes an opening positioned adjacent the proximal ends of the first and second arms, wherein the third diagnostic device moves through the opening.

In certain embodiments, the first arm has an opening at its distal end and the first diagnostic device moves through the opening. In additional embodiments, the second arm has an opening at its distal end and the second diagnostic device moves through the opening.

In some embodiments, the medical device further includes a control device that actuates at least one of the first, second and third diagnostic devices.

In certain embodiment, a fluid source is also provided positioned in at least one of the first arm and the second arm. In some of these embodiments, the fluid source delivers at least one of cleaning fluid, irrigation fluid and a therapeutic and/or diagnostic agent.

In some embodiments, the medical instrument further includes at least one cleaning device for cleaning at least one of the first, second and third diagnostic devices, wherein the at least one cleaning device is removably positioned inside the working device.

In certain embodiments, the medical instrument further includes at least one additional arm positioned between the first and second arms, wherein the third diagnostic device is movable between a first position wherein it extends between the first arm and the at least one additional arm and a second position wherein it extends between the second arm and the at least one additional arm.

A method of performing a medical therapeutic and/or diagnostic procedure is also provided, including inserting a medical instrument into a bodily cavity, the medical instrument comprising a shaft with a longitudinal axis and a working member at a distal end of the shaft, wherein the working device has a first arm and a second arm, wherein each of the first and second arms have a proximal end and a distal end, and wherein at least one of the first and second arms pivots relative to the longitudinal axis of the shaft, a first diagnostic device positioned in the first arm adjacent the distal end of the first arm, a second diagnostic device positioned in the second arm adjacent the distal end of the second arm, and a third diagnostic device positioned adjacent the proximal ends of the first and second arm, visualizing surrounding tissue via at least one of the first, second and third diagnostic devices, and actuating the working device to perform the procedure.

In some embodiments, the step of actuating the working device includes cutting bodily tissue.

In certain embodiments, the method further includes the step of measuring at least one characteristic of bodily tissue via at least one of the first, second and third diagnostic device.

In some embodiments, the working device further comprises at least one leaning device, and wherein the method further comprises the step of cleaning at least one of the first, second and third diagnostic device by displacing it through the at least one cleaning device.

In certain embodiments, the step of actuating the working device comprises actuating the first arm to contact and move a first layer of tissue and actuating the second arm to contact and move a second layer of tissue, such that first and second layers of tissue are moved away from each other.

Other objects of the invention and its particular features and advantages will become more apparent from consideration of the following drawings and accompanying detailed description.

### BRIEF DESCRIPTION OF THE DRAWINGS

FIG. 1A is a perspective view of a medical instrument with embedded imaging and/or diagnostic systems in accordance with the invention.

FIG. 1B is a cross-sectional view of the medical instrument of FIG. 1A, taken along the line “1B-1B”.

FIG. 2 is side view of a proximal end of the medical instrument of FIG. 1A, showing an actuation device.

FIG. 3A is an exploded perspective view of a camera used in the medical instrument of FIG. 1A.

FIG. 3B is an exploded side view of a camera used in the medical instrument of FIG. 3A.

FIGS. 4A and 4B illustrate the medical instrument of FIG. 1A being used in a bodily cavity.

FIGS. 5A and 5B illustrate a method of using at least one cleaning member of the medical instrument of FIGS. 4A and 4B.

FIG. 6 illustrates a method of using the medical instrument of FIG. 1A to deliver electrical current to tissue.

FIG. 7 illustrates a method of using the medical instrument of FIG. 1A to separate tissue layers.

FIG. 8 illustrates is a perspective view of a medical instrument of FIG. 1A with an additional arm.

### DETAILED DESCRIPTION OF THE INVENTION

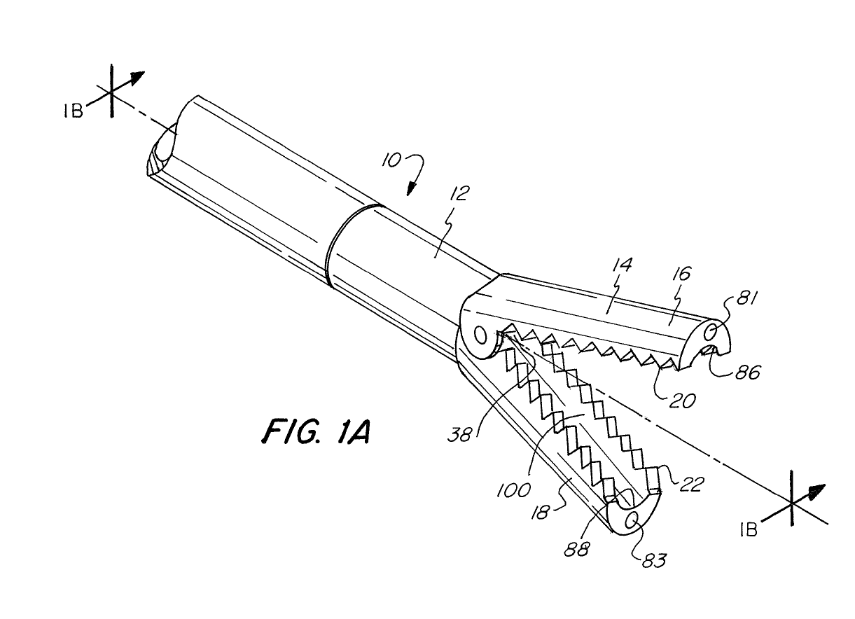

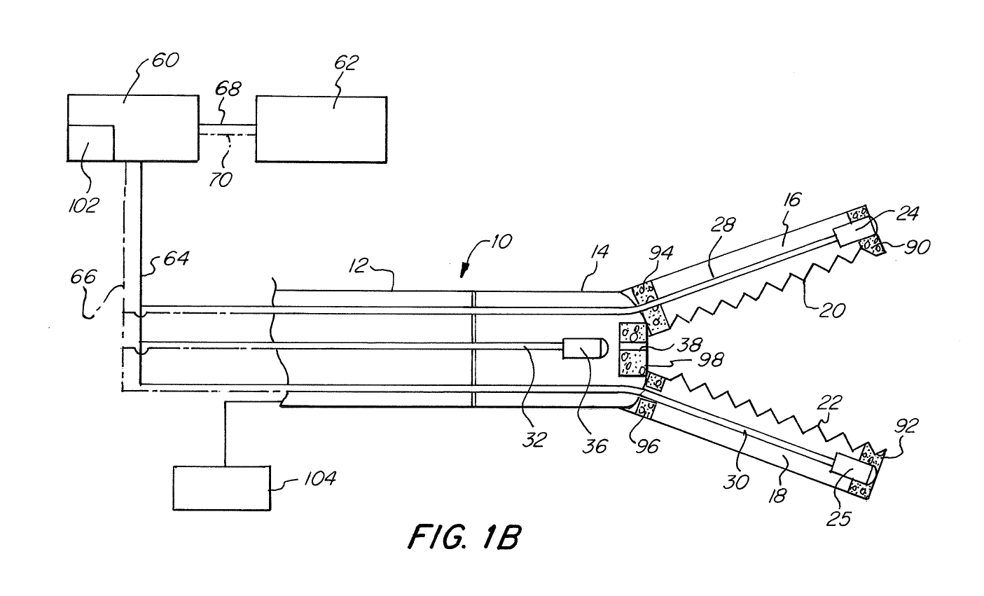

The basic components of one exemplary embodiment of a medical instrument with embedded imaging and/or diagnostic systems in accordance with the invention are illustrated in FIGS. 1A and 1B. As used in the description, the terms “top,” “bottom,” “above,” “below,” “over,” “under,” “above,” “beneath,” “on top,” “underneath,” “up,” “down,” “upper,” “lower,” “front,” “rear,” “back,” “forward” and “backward” refer to the objects referenced when in the orientation illustrated in the drawings, which orientation is not necessary for achieving the objects of the invention.

As shown in FIG. 1A, the medical instrument (10) includes an elongated shaft (12) having one or more lumens therein. The elongated shaft (12) may be constructed from any suitable rigid or semi-rigid material, such as, for example, stainless steel, titanium, polyether amide (PEBA), Pebax®, polyether ether ketone (PEEK), or polyurethane. The elongated shaft (12) is sized such that it can accommodate all of the components discussed below, and at the same time be capable of being introduced into a bodily cavity through a lumen of a catheter, working channel, or trocar, over a guide wire, or through a working channel of another medical instrument. It is understood that the medical instrument (10) can also be introduced into a patient's body via a percutaneous incision or via a natural orifice. In some embodiments, the medical instrument is inserted via a stereotactic instrument, which uses a 3D coordinate system to locate small targets inside a patient's body.

A working device (14) is positioned at a distal end of the elongated shaft (12). In some embodiments, the working device (14) is permanently attached to the elongated shaft (12). In additional embodiments, the working device (14) is detacheably connected to the distal end of the elongated shaft (12). The working device (14) is attached to the distal end of the elongated shaft (12) by any suitable attachment mechanism, such as a screw on or a snap-fit connector. It is understood that in the embodiment where the working device is a separate detachable unit, it may be attached to a distal end of any existing shaft device, such as a catheter.

In the embodiment illustrated in FIG. 1A, the working device (14) is a cutting device. The cutting device (14) includes an upper jaw (16) and a lower jaw (18) positioned on the distal end of the elongated shaft (12). The upper jaw (16) and the lower jaw (18) are hinged together so that they can be opened or closed to bite, grip or cut tissue. Examples of materials that can be used to construct the cutting device (14) include polymers such as PEEK, stainless steel, titanium, titanium alloys, etc.

In the embodiment shown in this figure, the edges of upper jaw and lower jaw are provided with a series of cutting teeth (20, 22). In other embodiments, the edges of the upper jaw (16) and the lower jaw (18) may be provided with sharp edges, blunt gripping teeth, etc. depending upon desired application. The upper and lower jaws (16, 18) may have any desirable shape. For example, the upper and lower jaws may be semi-circular, circular, square, ovoid, or trapezoidal in shape as viewed from their distal end.

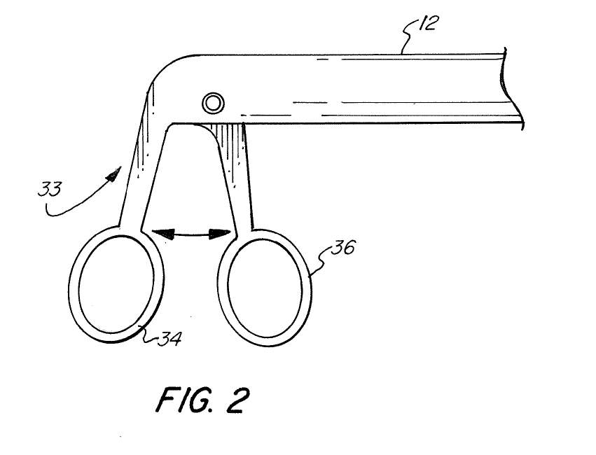

As shown in FIG. 2, a proximal end of the elongated shaft (12) includes a scissor-like actuation device (33) to control the movement of the upper jaw (16) and/or the lower jaw (18). The actuator device (33) includes two rings (34, 36) for insertion of a user's two fingers, preferably a thumb and an index finger. The elongated shaft (12) further includes one or more pull wires (not shown) that connect the upper jaw (16) and/or the lower jaw (18) to the actuation device (33). When the actuation device (33) is moved, the pull wires transmit the movement to the upper jaw (16) and the lower jaw (18) causing them to open or close. It is understood that any other suitable actuation device may be used in accordance with the present invention, including, for example, a trigger, paddle, lever, etc. The actuation device (33) enables linear, rotational, and/or flexional movement of the upper and lower jaws or arms (16, 18). It is further noted that actuation of the arms may be performed mechanically, pneumatically, robotically, and/or virtually/remotely, as further discussed below.

The upper and lower jaws (16, 18) are hollow inside and, in some advantageous embodiments, include openings at their distal ends. Furthermore, in certain advantageous embodiments, the cutting device (14) includes an opening (38) positioned at the base of the upper and lower jaws (16, 18) where they connect to each other. These openings may be used to introduce diagnostic devices, as discussed in more detail below. Additionally, these openings may be used for an access device, such as a guidewire. For example, a guidewire is passed through the lumen of the elongated shaft (12) out of the opening (38). In another embodiment, the guidewire passes through an opening located on either the upper jaw (16) or the lower jaw (18). This enables the cutting device to be advanced over an access device, such as a guidewire, to access a target anatomical region.

The cutting device (14) may also include a sample container or a storage compartment (100) inside one or both of the hollow upper and lower jaws (16, 18). Such design is particularly useful for performing biopsy procedures. Once a tissue sample is severed by the jaws, the tissue sample is placed in the storage compartment (100), the device is withdrawn from the patient's body and the tissue sample is retrieved from the compartment for analysis. In other advantageous embodiments, once the upper and lower jaws (16, 18) are closed, the severed tissue sample is simply held between the jaws until it is withdrawn from the patient's body.

Referring back to FIG. 1B, the cutting device (14) also includes a first diagnostic device (24) positioned adjacent the distal end of the upper jaw (16) and a second diagnostic device (25) positioned adjacent the distal end of the lower jaw (18). Furthermore, as shown in FIG. 1B, the cutting device includes a third diagnostic device (36) positioned inside the elongated shaft (12) adjacent the opening (38) between the upper and lower jaws (16, 18). Each of the first, second and third diagnostic devices (24, 25, 36) may comprise an imaging device.

Each of the diagnostic devices (24, 25, 36) is connected to a wiring harness (28, 30, 32 respectively). The harness enables data transmission between the devices and a processor, and provides illumination, power, and energy to the devices (24, 25, 36).

The first, second, and third imaging and/or diagnostic devices (24, 25, 26) comprise any desirable imaging and/or diagnostic device. In one embodiment described in more detail below, the imaging devices (24, 25, 36) comprise a camera. In additional embodiments, the imaging and/or diagnostic devices (24, 25, 36) comprise a probe or a sensor capable of detecting and/or measuring various characteristics of bodily tissues, including for example, temperature, fluid pressure, fluid density, fluid velocity, element content (e.g., oxygen or carbon content), physiologic dynamics, hemodynamics, molecular dynamics, etc. The sensors can also include therapeutic functionalities, e.g. point contact laser, etc. Any suitable sensor or probe type may be used in accordance with the present invention. It is understood that a combination of the cameras and probe/sensor devices may be used as well. For example, the first and second imaging and/or diagnostic device (24, 25) may comprise probes/sensors, and the third imaging and/or diagnostic device (36) may comprise a camera.

The camera may comprise any imaging device suitable for viewing the target area, such as a coherent fiber bundle or appropriate optical element and lens assembly in conjunction with an imaging sensor (e.g., CMOS, CCD), having a sufficiently small outer diameter, preferably about 0.75 mm-2.5 mm, and more preferably about 1 mm or less.

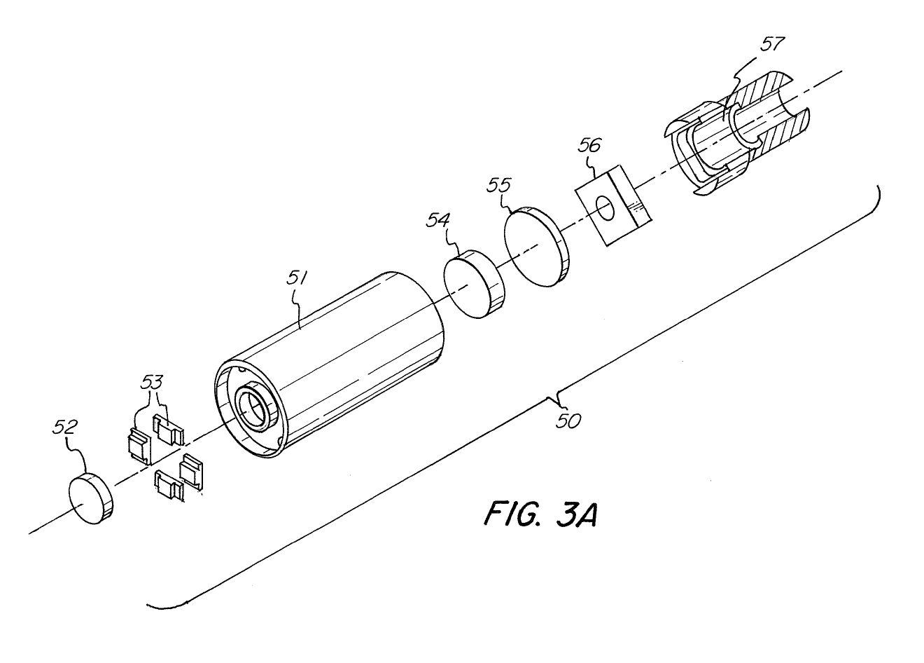

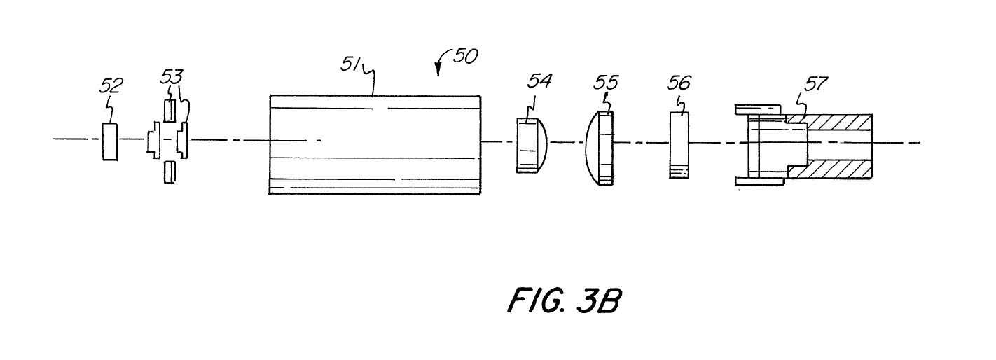

One advantageous camera embodiment is illustrated in FIGS. 3A and 3B. The camera (50) includes a camera housing (51) that houses all camera components. The housing (51) is made with any suitable material, such as plastic or metal, and has any desired shape and size. The camera also includes one or more lens positioned in the housing. In the embodiment shown in these figures, the camera includes two plano-convex lenses (54) and (55) positioned opposite of each other such that the convex sides of the lenses are facing each other. It is understood that any other lens type and arrangement may be used in accordance with the present invention, as desired.

The camera (50) further includes an imaging sensor (56) positioned proximally from the lens (54) and (55). Any type of imaging sensor may be used. The imaging sensor (56) is coupled a sensor mount (57) to fixate the sensor inside the housing. In one advantageous embodiment, a CMOS sensor is used. The housing (51) also has one or more illumination devices (53), e.g. LEDs, lasers, and/or fiber optic cables, positioned distally from the lens. It is understood than other types of illumination devices may be used. Furthermore, illumination devices that are separate from the camera may also be utilized in accordance with the present invention.

The illumination devices emit various types of light, depending on desired application. For example, the illumination devices may emit ambient light, visible spectrum light, ultraviolet light, infrared light, near infrared light, etc. A distal end of the housing (51) has a screen (52) that seals the distal end of the housing to protect the camera components positioned in the housing.

It is understood that the camera design illustrated in FIGS. 3A and 3B is only exemplary and that any other camera design may be used with the system of the present invention. The camera is coupled to an actuator that enables a linear, rotational or angular movement of the camera, as described in more detail below, to provide a larger angle of view. The actuation of the camera may be mechanical or software based.

The imaging and/or diagnostic devices of the present invention allow examination of the bodily anatomy using light of various spectrums and various wavelengths. This allows for detection, visualization and characterization of various tissues, structures, and molecular compounds that may be present in the body, which in turn lead to diagnosis of various diseases. This is due to the fact that various tissues and structures that may be present in the body absorb and/or deflect light of various spectra and/or wavelengths in different ways. Analysis of the light scattering thereby provides information about particular tissues and structures. The system of the present invention also allows for detection and characterization of changes in body anatomy over time, which may be caused by various diseases. The system is capable of measuring color saturation of the light emitted onto the target tissues and also measures scattering of light deflected from the target tissues in the body.

As noted above, the imaging devices of the present invention may utilize a plurality of illumination devices or light sources. In some embodiments, all of the light sources emit light of the same spectrum/wavelength. In additional embodiments, each of the plurality of light sources emits light of a different spectrum/wavelength than the light emitted by other light sources. This allows for detection and characterization of various structures and conditions inside the body, as described above. The illumination devices or light sources also provide necessary illumination to the surgical site to assist the physician in performing the therapeutic or diagnostic medical procedure.

The first and second imaging devices (24, 25) enable visualization of the surrounding anatomy in 3D format by overlapping the images received from each device. Furthermore, the images received from the first and second imaging devices (24, 25) may also be combined with the images received from the third imaging device (36) to enable visualization of the surrounding tissue in a 4D format. It is understood that more than three imaging devices may be used in accordance with the present invention, which enable visualization of the target tissue in 5D and higher formats.

Referring back to FIG. 1B, the medical instrument (10) further includes a processor (60) coupled to the first, second and third imaging and/or diagnostic devices (24, 25, 36) for receiving and processing data captured by the devices. Any suitable processor may be used in accordance with the present invention. For example, the processor (60) may be a personal computer. The digital image data or other data captured by the imaging and/or diagnostic devices (24, 25, 36) is transmitted to the processor for analysis and for creating images and other information that is displayed to the physician. One of the techniques that are may be utilized to process the captured digital data is spectroscopy, which analyzes interaction between matter and radiated energy. By utilizing spectroscopy techniques, it is possible to digitally process spectrums and wavelengths reflected from bodily tissues to detect and characterize various elements present in the tissue.

In one advantageous embodiment, the processor (60) is connected to the devices (24, 25, 36) via a cable or wired connection (64). In additional advantageous embodiments, the processor (60) is connected to the devices (24, 25, 36) via a wireless, e.g. cellular or satellite, connection (66), which is particularly desirable if a physician is located remotely from a patient undergoing the medical procedure. For example, the system of the present invention may be used by physicians located in field conditions, such as on a battlefield, wherein there is no time or accessibility to analyze the data captured by the devices (24, 25, 36). The physicians utilize the devices (24, 25, 36) to capture various data and then send it wirelessly to remote locations for analysis. In further advantageous embodiments, the captured data may be stored in cloud storage, meaning that the digital data is stored in logical pools, with the physical storage typically spanning across multiple servers managed by a hosting company. This way, the data may be easily accessed from any location connected to the cloud storage, such as physicians' and patients' personal computers, tablets and smartphones.

The medical instrument (10) may also include a control device (102) for actuating the imaging and/or diagnostic devices (24, 25, 36) and/or the cutting device (14). In some embodiments, the control device may be positioned at a proximal end of the elongated shaft (12). In additional embodiments, the control device (102) may be positioned remotely from the patient and may be integral or connected to the processor (60). The control device (102) may be connected to the imaging and/or diagnostic devices (24, 25, 36) and/or the cutting device (14) via a wired or wireless connection. This is particularly advantageous, for example, in robotically-assisted surgery, wherein a surgeon typically operates a controller to remotely control the motion of various medical devices affixed to robotic arms positioned at a surgical site. The control device is in a location that may be remote from the patient (e.g., across the operating room, in a different room or a completely different building from the patient). The control device typically includes one or more hand input devices, such as handheld wrist gimbals, joysticks, exosceletal gloves, handpieces, etc., coupled to the robotic arms holding the medical devices.

Furthermore, the imaging and/or diagnostic devices (24, 25, 26) and/or the processor (60) may be connected to an external storage device, a removable storage device, and/or to an internet port. The data captured by the imaging and/or diagnostic devices (24, 25, 36) is stored on the storage device and may be later retrieved by a user. In other advantageous embodiments, the processor (60) may have an internal storage device. Any suitable storage device may be used in accordance with the present invention.

In some embodiments, the data collected by the imaging and/or diagnostic devices (24, 25, 36) is compressed before it is transmitted to the processor for processing or storage. In other words, the data is encoded using fewer bits than the originally captured data to reduce resource usage, such as data storage space or transmission capacity. Once the compressed data is received by the processor, it is decompressed before it is displayed to the user to maintain the original quality of the captured images.

The medical instrument (10) may further include a display (62) coupled to the processor (60) via a cable connection (68) or via a wireless connection (70). The display (62) receives data processed by the processor (60) and displays the image of the person's anatomy in 2-D format and 3-D format to a physician. Any suitable type of a display may be used in accordance with the present invention.

As shown in FIGS. 1A and 1B, the upper and lower jaws (16, 18) of the cutting instrument (14) each have an opening (81, 83) at their distal end. When the device is in use, the imaging and/or diagnostic devices (24, 25) are extended out of the openings (81, 83) to visualize and/or collect data from the surrounding tissue. There is also an opening (38) at the base of the upper and lower jaws (16, 18), through which the imaging and/or diagnostic device (36) may be extended. In advantageous embodiments, the distal ends of the upper and lower jaws (16, 18) each have a recess (86, 88), as shown in FIG. 1A, shaped such that there is a channel provided for the device (36) to extend out of the shaft (12) when the jaws are in the closed position.

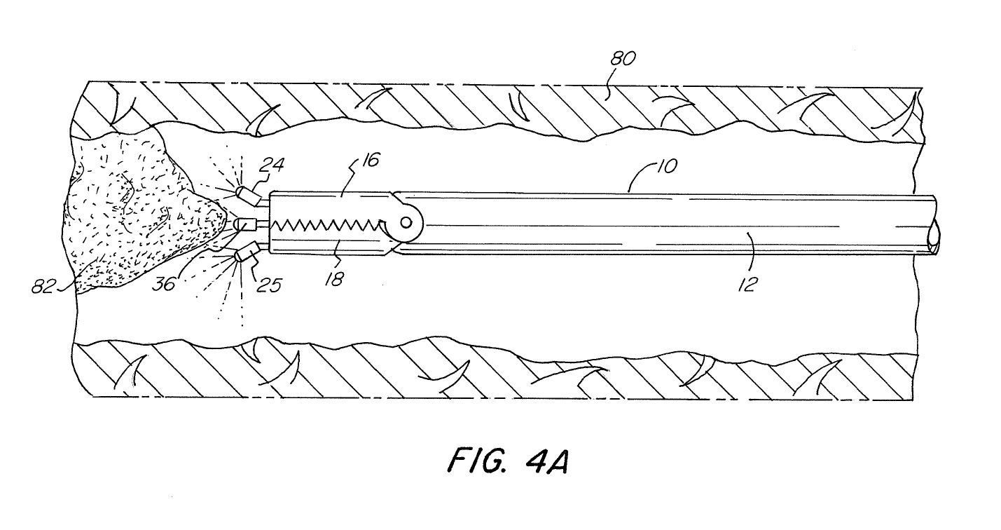

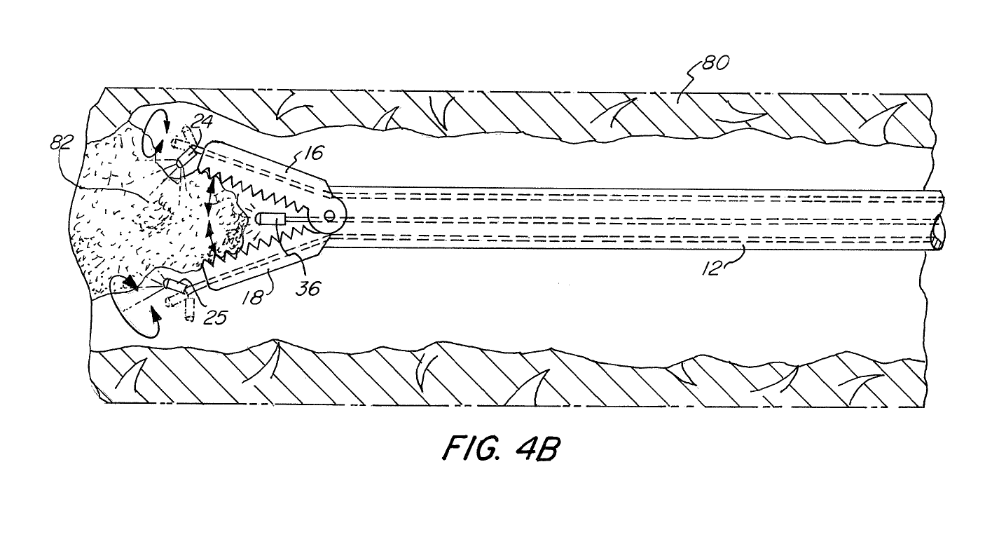

The method of using the device (10) in a patient's body is illustrated in FIGS. 4A and 4B. As shown in FIG. 4A, the device (10) is inserted into a bodily cavity (80), such as a blood vessel or an airway, until it reaches a target tissue site (82), for example, a tumor. During the insertion of the device (10), the upper and lower jaws (16, 18) are in a closed position to facilitate the insertion and to prevent possible injury to surrounding tissues. In some embodiments, a protective sheath may be placed over the elongated shaft (12) during the insertion of the device (10) to protect bodily tissues. In additional embodiments, the elongated shaft may be inserted into the bodily cavity over a guide wire positioned in a lumen of the elongated shaft (12) and extended out of the opening (38) in the shaft. In further embodiments, the elongated shaft (12) may have steering capabilities such that it can be maneuvered into the bodily cavity by a physician.

During the insertion of the device (10) into the bodily cavity (80) it is desirable to be able to visualize the surrounding tissue to ensure the proper positioning of the device adjacent the target tissue (82). As illustrated in FIG. 4A, during the insertion, the imaging devices (24, 25, 36) are extended out of the distal end of the elongated shaft (12) to visualize the tissue in front and around the device (10). In advantageous embodiments, the devices (24, 25, 36) are provided with illumination sources to provide necessary illumination of the tissue. The devices (24, 25, 36) are translated longitudinally such that they extend out of the distal end of the shaft (12) to a desired degree. One or more of the devices (24, 25, 36) is also actuated rotationally to bend in different directions, as shown in this figure, to visualize tissue around the shaft and obtain different angles of view. The actuation of the devices (24, 25, 36) is accomplished by any suitable actuation device, for example, a mechanical, pneumatic and/or electro-magnetic device.

Once the device (10) is positioned at the desired tissue site, the working device (14) is actuated to perform the desired medical procedure. In the embodiment shown in FIG. 4B, the upper and lower jaws (16) and (18) are actuated to resect tissue (82). During this procedure, the imaging/diagnostic devices (24, 25, 36) are used to visualize the procedure and the surrounding tissue to facilitate the accuracy of the procedure. In some embodiments, a resected tissue sample may be captured in a container inside one or both of the hollow upper and lower jaws (16, 18), withdrawn from the patient's body and then retrieved from the container for analysis. In other embodiments, the tissue sample may be simply held between the jaws until it is withdrawn from the patient's body. In yet further embodiments, the tissue sample may be held between the jaws while it is being diagnosed by the imaging/diagnostic devices (24, 25, 36).

The devices (24, 25, 36) may also collect data regarding various characteristics of bodily tissues, including, for example, temperature, fluid pressure, fluid density, fluid velocity, oxygen content, etc. For example, during treatment of cancerous tumors, it may be helpful to know the oxygen content of the tumor tissue to be able to more effectively treat the tumor. This data is collected by a probe or a sensor positioned on at least one of the imaging and/or diagnostic devices (24, 25, 36). The data collected by the devices (24, 25, 36) is transmitted to the processor (60) for processing and display to the physician.

Referring back to FIG. 1B, the device (10) further includes at least one lens cleaning member positioned inside the arms of the working device (14). In the embodiment shown in this figure, the upper jaw (16) has a cleaning member (94) positioned adjacent its base and a cleaning member (90) positioned adjacent its distal end. Similarly, the low jaw (18) has a cleaning member (96) adjacent its base and a cleaning member (92) adjacent its distal end. It is understood that the less or more cleaning members may be positioned inside the lower and upper jaws. It is further understood that the cleaning members may be positioned at any desired location along the jaws (16, 18). The working device (14) further includes a cleaning member (98) positioned adjacent the opening (38).

The cleaning members may comprise any suitable material, such as, e.g., polyamide. For example, in some embodiments, the cleaning members comprise a conduit and a plurality of flexing flaps extending into and at least partially occluding the conduit. When the devices (24, 25, 36) are moved through the conduit, the flexing flaps contact the lens of the devices, thereby wiping off any debris or residue from the lens. In other embodiments, the cleaning members comprise tubular pieces of flexible porous material, such as a sponge, with a conduit that extends through the tubular piece. The devices (24, 25, 36) are cleaning as they are moved through the conduit by contact with the flexible porous material. In yet further embodiments, the cleaning members comprise fibrous material, such as yarn, that has been weaved under tension and then released such that the yarn tangles, creating a textile bundle. When the devices (24, 25, 36) are moved through the bundle, some of the yarns in the bundle are displaced by the devices, which facilitates whipping and cleaning of the device lens. Examples of the cleaning members that may be used in connection with the device of the present invention are described in U.S. Patent Publication No. 2012/0238816 to Gunday et al., the disclosure of which is incorporated here in its entirety.

It should be noted that any other suitable flexible material may be used for the cleaning members (90, 92, 94, 96, 98). In some embodiments, the cleaning members may be made of bio-degradable materials. The cleaning members may be removable, cleanable and/or disposable. In some advantageous embodiments, the cleaning members possess surface tension and/or absorption characteristics that facilitate retention of water or cleaning fluid in the material of the cleaning members.

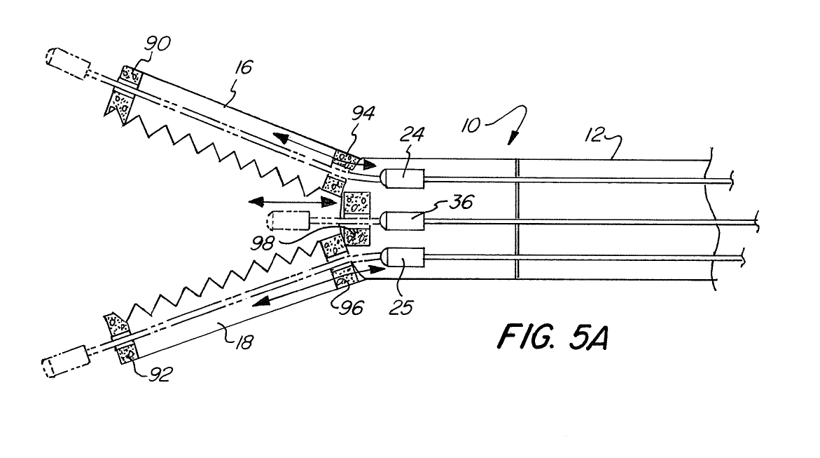

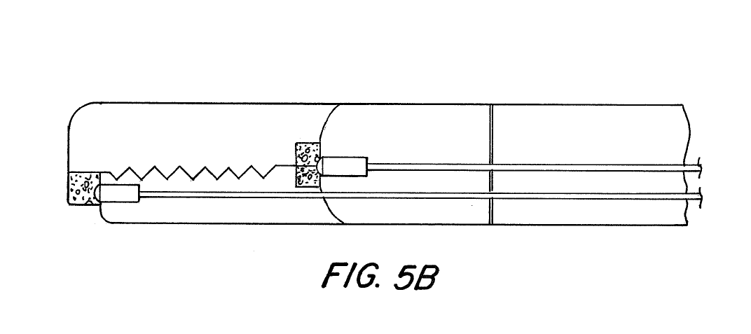

As shown in FIG. 5A, when the lens of the imaging/diagnostic devices (24, 25, 36) becomes fouled or fogged, the devices are cleaned by retracting them back through the conduits in the cleaning members (90, 92, 94, 96, 98) and then extending them back out. As the devices (24, 25, 36) are moved through the cleaning members, the distal tips of the devices push through the flexible material of the cleaning members, and any debris trapped on the lens are wiped off by the cleaning members. The devices (24, 25, 36) may be actuated back and forth through the cleaning members as many times as needed, until the lens is completely cleaned off.

In another embodiment shown in FIG. 5B, the imaging/diagnostic devices (24, 25, 36) do not need to be moved through the cleaning members in order to clean the lens. Instead, the upper arm (16) has a lip (155) that extends over the distal end of the second arm (18) when the arms are in a closed position. The lip (155) has a cleaning member (90) attached to its distal end. When the upper arm (16) is closed over the lower arm (18), the cleaning member (90) wipes off the lens of the imaging device (25) positioned in the lower arm (18). It is understood that, in other embodiments, the lower arm (18) may instead have a lip with a cleaning member such that the lip extends over the distal end of the upper arm (16) when the arms are in a closed position and cleans off the lens of the imaging device positioned in the upper arm.

The upper arm (16) and the lower arm (18) may also have cleaning members (94, 96) placed adjacent the proximal ends of the arms. When the upper and lower arms (16, 18) are moved towards a closed position shown in FIG. 5B, the cleaning members (94, 96) wipe off the lens of the imaging device (36) extended through the opening between the arms, thereby cleaning it.

In some advantageous embodiments, a cleansing solution is used to further assist in cleaning the lens of the devices (24, 25, 36). Any suitable cleansing solution, such as saline solution, glycol solution, alcohol solution, water, any combination thereof, or any other biocompatible fluid may be used in accordance with the present invention. In some embodiments, the cleansing solution is supplied though a fluid lumen in the working device (14) connected to a fluid source (104), as shown in FIG. 1B. The fluid lumen extends through the elongated shaft (12) and then into each of the upper and lower jaws (16, 18). The cleansing solution is used to saturate the cleaning members (90, 92, 94, 96, 98) to enhance the cleaning of the devices (24, 25, 36). Additionally, the cleansing solution may be provided to inside of the hollow working device (14) to rinse the devices (24, 25, 36) as they move through the cleaning members.

In some embodiments, the fluid lumens may also be used to provide irrigation to the surgical site. Furthermore, the fluid lumens may be used to deliver various therapeutic and/or diagnostic agents and fluids to the target tissue. The fluids may be also delivered via a nebulizing tip connected to a distal end of each fluid lumen.

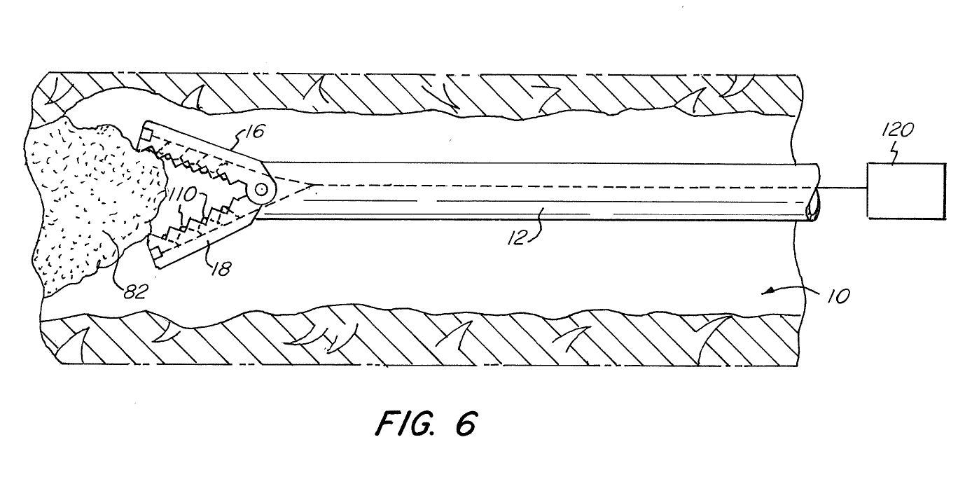

The medical instrument (10) of the present invention may also include an electrocautery device. Electrocautery devices are typically used for surgical dissection and hemostasis and function by delivering heat generated by high-voltage, high-frequency alternating current passed through an electrode. As illustrated in FIG. 6, the medical instrument (10) includes one or more electrically conductive members (110) positioned in one or both of the upper and lower jaws (16, 18). The electrically conductive members (110) may be electrodes or any other suitable devices. The electrically conductive members (110) extend through the elongated shaft (12) and are coupled to a source of electrical current (120) positioned at the proximal end of the instrument (10). When in use, the electrical current is delivered from the source (120) to tissue (82) through the electrodes (110) positioned at the distal end of the instrument to cauterize the tissue.

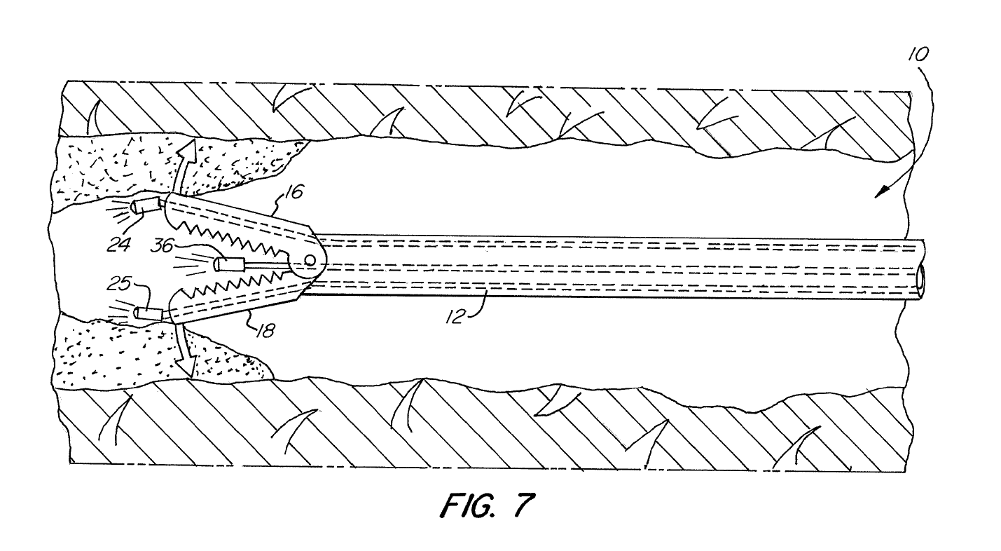

FIG. 7 illustrates another use of the medical instrument (10) of the present invention to perform a blunt dissection procedure. This procedure involves a separation of tissues by blunt instruments during various surgical procedures. During the procedure, the medical instrument (10) in a closed position is wedged between two layers of tissue (130) and (140). Then, the upper and lower arms (16, 18) are opened such that each of the arms moves one of the tissue layers away from the other layer to open up space between the layers. As this is performed, the opening between the tissue layers is visualized by one or more of the cameras (24, 25, 36).

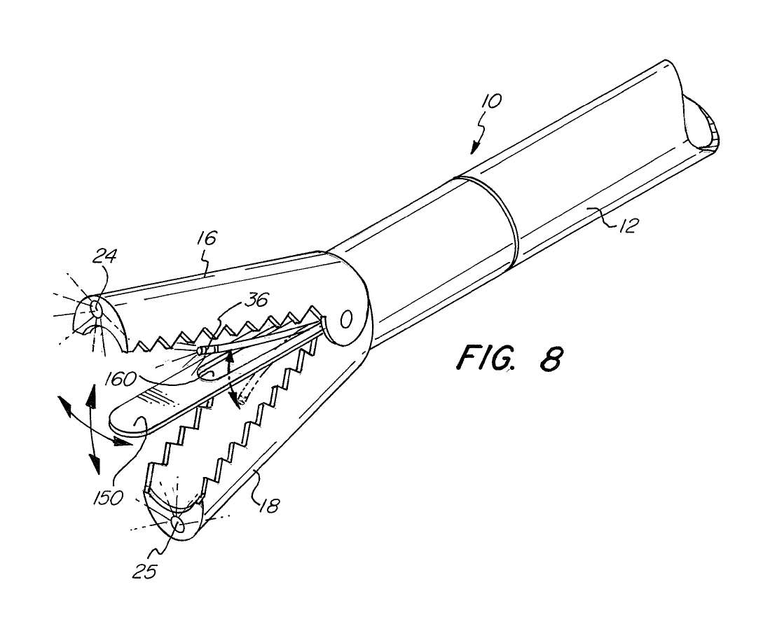

In some embodiments, the medical instrument of the present invention may have more than two arms/jaws. For example, as shown in FIG. 8, the medical instrument (10) has an upper arm (16), a lower arm (18) and a middle arm or a tongue (150) positioned between the upper and lower arms. The tongue (150) may have a flattened, half-moon or any other desired shape and is made with any suitable material, such as, e.g., PEEK, stainless steel. titanium, titanium alloys, etc. It may also have a cutting or grasping surface similar to the cutting or grasping surface of the upper and/or lower arms. The tongue is used to perform a variety of desired procedures, such as cutting, grasping, or lifting tissue, etc. It is understood that any number of additional arms may be positioned between the upper and lower arms.

The tongue (150) is coupled to an actuation device that enables linear, rotational, and/or flexional movement of the tongue. In some embodiments, the actuation device that actuates the tongue (150) may be the same actuation device used to actuate the upper and/or lower arms. In other embodiments, the tongue and the lower and upper arms are actuated separately from each other. Any suitable type of actuation device is used to actuate the tongue, including mechanical, pneumatic, robotic, and/or virtual actuation devices.

A proximal end of the tongue (150) has a cut-out portion (160). The diagnostic/imaging device (36) is positioned in the cut-out portion (160) such that it can be actuated to extend between the tongue (150) and the upper arm (16) or between the tongue (150) and the lower arm (18). This allows for visualization of both the space between the tongue and the upper arm and the space between the tongue and the lower arm. The device (36) is capable of linear, rotational and/or flexional movement and is actuated by any suitable mechanism, such as, e.g., a pull wire, pneumatic device, piezoelectrics, gimbal wrist, etc. It is understood that the medical instrument (10) may include two or more diagnostic/imaging devices positioned between the tongue, the upper arm and the lower arm.

The present invention may utilize a plurality of medical instruments to perform a plurality of medical procedures simultaneously or sequentially. The plurality of medical instruments are also used to provide a combined image of target anatomy by visualizing different angles of the anatomy, as well as providing a view from inside the anatomy. Furthermore, the plurality of instruments provide combined data from various sensors positioned on each instrument. These features allow a surgeon to collect data to understand tissue integrity, bioelectrical integrity, electrical fields, disease state and any other desirable characteristics of the anatomy.

The plurality of medical instruments of the present invention are also useful in image guided or stereotaxic surgery. The instruments are oriented to provide different angle images of the surgical site to facilitate the imaging of the site by the surgeon. Additionally, the instruments may include location sensors to provide a 3D coordinate system to locate small targets inside a patient's body.

It should be understood that the foregoing is illustrative and not limiting, and that obvious modifications may be made by those skilled in the art without departing from the spirit of the invention. Accordingly, reference should be made primarily to the accompanying claims, rather than the foregoing specification, to determine the scope of the invention.

What is claimed is:

1. A medical instrument, comprising:
a shaft having a proximal end, a distal end and a longitudinal axis;
a working member at the distal end of the shaft and comprising a first arm and a second arm, wherein each of the first and second arms comprises a proximal end and a distal end, and wherein at least one of the first and second arms pivots relative to the longitudinal axis of the shaft;
a first diagnostic device positioned in the first arm adjacent the distal end of the first arm;
a second diagnostic device positioned in the second arm adjacent the distal end of the second arm; and
a third diagnostic device positioned adjacent the proximal ends of the first and second arm.

2. The medical instrument of claim 1, wherein at least one of the first arm and the second arm comprises a cutting surface.

3. The medical instrument of claim 1, wherein at least one of the first arm and the second arm comprises a grasping surface.

4. The medical instrument of claim 1, wherein at least one of the first arm and the second arm comprises a storage compartment for retaining a tissue sample.

5. The medical instrument of claim 1, wherein said working member comprises an electrically conductive member coupled to a source of electrical current for delivering electric current to tissue.

6. The medical instrument of claim 1, wherein the working device is removably attached to the shaft.

7. The medical instrument of claim 1, further comprising an actuator positioned at the proximal end of the shaft that actuates the working device.

8. The medical instrument of claim 1, wherein at least one of the first, second and third diagnostic devices comprises a sensor that detects and measures at least one characteristic of bodily tissue.

9. The medical instrument of claim 1, wherein at least one of the first, second and third diagnostic devices comprises a camera.

10. The medical instrument of claim 9, wherein the camera comprises at least one lens and at least one imaging sensor.

11. The medical instrument of claim 10, wherein the imaging sensor comprises a CMOS sensor.

12. The medical instrument of claim 9, wherein at least one of the first, second and third diagnostic devices further comprises at least one illumination device positioned adjacent the camera.

13. The medical instrument of claim 12, wherein the at least one illumination device comprises a light source emitting light having a visible spectrum.

14. The medical instrument of claim 12, wherein the at least one illumination device comprises a light source emitting light having a non-visible spectrum.

15. The medical instrument of claim 1, further comprising a processor connected to the first, second and third diagnostic devices that receives and processes data captured by the diagnostic devices.

16. The medical instrument of claim 15, wherein the processor is connected to the first, second and third diagnostic devices via a wired connection.

17. The medical instrument of claim 15, wherein the processor is connected to the first, second and third diagnostic devices via a wireless connection.

18. The medical instrument of claim 1, wherein the working device comprises an opening positioned adjacent the proximal ends of the first and second arms, wherein the third diagnostic device moves through the opening.

19. The medical instrument of claim 1, wherein the first arm comprises an opening at its distal end and the first diagnostic device moves through the opening.

20. The medical instrument of claim 1, wherein the second arm comprises an opening at its distal end and the second diagnostic device moves through the opening.

21. The medical instrument of claim 1, further comprising a control device that actuates at least one of the first, second and third diagnostic devices.

22. The medical instrument of claim 1, further comprising a fluid source positioned in at least one of the first arm and the second arm.

23. The medical instrument of claim 22, wherein the fluid source delivers at least one of cleaning fluid, irrigation fluid and a therapeutic and/or diagnostic agent.

24. The medical instrument of claim 1, further comprising at least one cleaning device for cleaning at least one of the first, second and third diagnostic devices, wherein the at least one cleaning device is removably positioned inside the working device.

25. The medical instrument of claim 1, further comprising at least one additional arm positioned between the first and second arms, wherein the third diagnostic device is movable between a first position, wherein it extends between the first arm and the at least one additional arm, and a second position wherein it extends between the second arm and the at least one additional arm.

26. A method of performing a medical therapeutic and/or diagnostic procedure, comprising:
inserting a medical instrument into a bodily cavity, said medical instrument comprising a shaft with a longitudinal axis and a working member at a distal end of the shaft, wherein the working device comprises:
a first arm and a second arm, wherein each of the first and second arms comprises a proximal end and a distal end, and wherein at least one of the first and second arms pivots relative to the longitudinal axis of the shaft,
a first diagnostic device positioned in the first arm adjacent the distal end of the first arm,
a second diagnostic device positioned in the second arm adjacent the distal end of the second arm, and
a third diagnostic device positioned adjacent the proximal ends of the first and second arm;
visualizing surrounding tissue via at least one of the first, second and third diagnostic devices; and
actuating the working device to perform the procedure.

27. The method of claim 26, further comprising the step of measuring at least one characteristic of bodily tissue via at least one of the first, second and third diagnostic device.

28. The method of claim 26, wherein the working device further comprises at least one cleaning device, and wherein the method further comprises the step of cleaning at least one of the first, second and third diagnostic device by displacing it through the at least one cleaning device.

29. The method of claim 26, wherein the step of actuating the working device comprises actuating the first arm to contact and move a first layer of tissue and actuating the second arm to contact and move a second layer of tissue, such that first and second layers of tissue are moved away from each other.
